(19) United States
(12) Patent Application Publication
(10) Pub. No.: US 2017/0245898 A1
(43) Pub. Date: Aug. 31, 2017

(54) SPINAL IMPLANT SYSTEM AND METHODS OF USE

(71) Applicant: Warsaw Orthopedic, Inc, Warsaw, IN (US)

(72) Inventors: Jason M. May, Collierville, TN (US); William A. Rezach, Atoka, TN (US); Brian A. Butler, Atoka, TN (US)

(21) Appl. No.: 15/054,384

(22) Filed: Feb. 26, 2016

(51) Int. Cl. A61B 17/70

(52) U.S. Cl. CPC A61B 17/7037; A61B 17/7032

(57) ABSTRACT

A bone fastener comprises a first member defining an implant cavity and a groove configured for disposal of a band. The first member includes a part disposed with the implant cavity and engages with the band to provisionally fix the part relative to the first member. A second member is configured to penetrate tissue and includes a base that moves the part and engages the band to connect the members. Implants, systems, instruments and methods are disclosed.

### TECHNICAL FIELD

The present disclosure generally relates to medical devices for the treatment of spinal disorders, and more particularly to a surgical implant system including a bone fastener and a related method.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs such as vertebral rods are often used to provide stability to a treated region. Rods redirect stresses away from a damaged or defective region while healing takes place to restore proper alignment and generally support the vertebral members. During surgical treatment, one or more rods and bone fasteners can be delivered to a surgical site. The rods may be attached via the fasteners to the exterior of two or more vertebral members. This disclosure describes an improvement over these prior technologies.

### SUMMARY

In one embodiment, a bone fastener is provided. The bone fastener comprises a first member defining an implant cavity and a groove configured for disposal of a band. The first member includes a part disposed with the implant cavity and engages the band to provisionally fix the part relative to the first member. A second member is configured to penetrate tissue and includes a base that moves the part and engages the band to connect the members. In some embodiments, implants, systems, instruments and methods are disclosed.

In one embodiment; a method of assembly for a bone fastener is provided. The method comprises the steps of: engaging a part of an implant receiver with a band disposed within a groove of the implant receiver to provisionally fix the part relative to the implant receiver; and manually engaging a base of a screw shaft with the part to move the part and engage the band to connect the implant receiver with the screw shaft to comprise a bone fastener in a non-instrumented assembly.

### BRIEF DESCRIPTION OF THE DRAWINGS

The present disclosure will become more readily apparent from the specific description accompanied by the following drawings, in which:

FIG. 1 is a perspective view of components of one embodiment of a spinal implant system in accordance with the principles of the present disclosure;

FIG. 2 is a cross section view of components of one embodiment of the system shown in FIG. 1;

FIG. 3 is a perspective view of components of the system shown in FIG. 2;

FIG. 4 is a perspective view of components of the system shown in FIG. 2;

FIG. 5 is a cross section view of components of the system shown in FIG. 2;

FIG. 6 is a cross section view of components of the system shown in FIG. 2;

FIGS. 7, 8, 9 are cross section views of components of one embodiment of a spinal implant system illustrating assembly;

FIG. 10 is an end view of components of the system shown in FIG. 2;

FIG. 11 is an end view of components of the system shown in FIG. 2;

FIG. 12 is a side view of components of the system shown in FIG. 2;

FIG. 13 is a side view of components of the system shown in FIG. 2;

FIG. 14 is a side view of components of the system shown in FIG. 2;

FIG. 15 is a side view of components of the system shown in FIG. 2;

FIG. 16 is an end view of components of the system shown in FIG. 2;

FIG. 17 is an end view of components of the system shown in FIG. 1;

FIG. 18 is an end view of components of the system shown in FIG. 2;

FIG. 19 is an end view of components of the system shown in FIG. 2;

FIG. 20 is a perspective view of components of one embodiment of the system shown in FIG. 1;

FIG. 21 is a cross section view of components of the system shown in FIG. 20;

FIG. 22 is a perspective view of components of the system shown in FIG. 20;

FIG. 23 is a cross section view of components of the system shown in FIG. 20;

FIGS. 24, 25, 26 are cross section views of components of one embodiment of a spinal implant system illustrating assembly;

FIG. 27 is a cross section view of components of one embodiment of the system shown in FIG. 20;

FIG. 28 is a perspective view of components of the system shown in FIG. 27;

FIG. 29 is a side view of components of the system shown in FIG. 27;

FIG. 30 is a side view of components of the system shown in FIG. 27;

FIG. 31 is an end view of components of the system shown in FIG. 27;

FIG. 32 is a side view of components of the system shown in FIG. 27;

FIG. 33 is an end view of components of the system shown in FIG. 27; and

FIG. 34 is a side view of components of the system shown in FIG. 27.

### DETAILED DESCRIPTION

The exemplary embodiments of a surgical system and related methods of use disclosed are discussed in terms of medical devices for the treatment of musculoskeletal disorders and more particularly, in terms of a spinal implant system including a bone fastener. In one embodiment, the spinal implant system includes an implant comprising a bone fastener, such as, for example, a universal pedicle bone screw. In some embodiments, the spinal implant system includes a selectively coupled pedicle screw system that allows for operating room back-table assembly of a bone fastener without use of instrumentation.

In some embodiments, the spinal implant system comprises a modular screw system. In some embodiments, the spinal implant system comprises a modular screw system including a base component that is pre-assembled with a bone screw shaft. In some embodiments, the spinal implant system comprises a modular screw system including a base component that facilitates attachment of an implant receiver with a screw shaft in a robust connection. In some embodiments, the spinal implant system comprises a modular screw system including screw shaft assemblies and implant receiver/head assemblies that may be joined together during manufacturing or intra-operatively, such as, for example, during a surgical procedure in an operating room.

In some embodiments, a screw shaft assembly includes at least a bone screw and a screw base. In some embodiments, a base component may be assembled to the bone screw shaft using a force and/or interference fit. In some embodiments, a base component may be assembled or retained to the bone screw shaft using one or more retaining members, such as, for example, snap rings. In some embodiments, a base component may be assembled or retained to the bone screw shaft by displacing material of the base component, such as, for example, a swage or stake operation. In some embodiments, a head assembly includes a crown, a tulip and a snap ring. In some embodiments, a snap ring is engaged in a retaining groove in the tulip head and provisionally engaged to the crown. In some embodiments, a snap ring is engaged to a part, such as, for example, a sleeve that maintains the snap ring centered. In some embodiments, the present system is employed with a method of assembly such that during assembly the screw base drives and/or translates the crown or sleeve upwards to force the snap ring to expand and disengage the crown. In some embodiments, the method includes the step of engaging the screw shaft assembly with the head assembly such that the snap ring engages a retaining groove in the screw base and creates a permanent assembly of a bone fastener.

In some embodiments, the spinal implant system comprises a modular system that includes a head assembly having a tulip, a crown, a sleeve and a snap ring. In some embodiments, the spinal implant system comprises a modular system that includes a screw shaft assembly including a screw base having an indexing feature. In some embodiments, the screw shaft assembly includes a shaft having a head with flats for engagement to the indexing feature. In some embodiments, the screw shaft assembly includes a screw base that is rotatable relative to an implant receiver in a range of 0 through 360 degrees. In some embodiments, the screw shaft assembly includes a shaft having a head with flats for engagement to the indexing feature such that the screw shaft is prevented from rotation in a selected plane. In some embodiments, the screw shaft assembly includes a shaft having a head with flats for engagement to the indexing feature such that the screw shaft is rotatable in a selected plane. In some embodiments, the screw shaft assembly includes a shaft having a head with flats for engagement to the indexing feature such that the bone fastener has uniaxial planar movement. In some embodiments, the screw shaft assembly includes a shaft having a head with flats for engagement to the indexing feature such that the screw shaft can be positioned by rotating the screw base relative to the head assembly.

In some embodiments, the screw shaft assembly includes a biased angle screw base. In some embodiments, the biased angle screw base includes a relief notch. In some embodiments, the relief notch is configured for disposal of the screw shaft to provide the screw shaft with additional angulation and standard angulation with the screw shaft positioned outside of the relief notch. In some embodiments, the biased angle screw base is rotatable such that the relief notch can be positioned through an angular range of 0 through 360 degrees relative to an implant receiver to provide the screw shaft with selective additional angulation at a selected angle in the angular range.

In some embodiments, the present system is employed with a method of assembly including the step of initially engaging a screw shaft assembly with a head assembly such that the screw shaft assembly is disposed with a bore of the head assembly. In some embodiments, the method includes the step of expanding a snap ring such that the snap ring is expanded by the screw shaft assembly forcing a crown upwards in the head assembly. In some embodiments, this configuration allows the crown to disengage from the snap ring. In some embodiments, the method includes the step of collapsing the snap ring such that as the screw shaft assembly travels into the head assembly, the snap ring collapses in a retaining groove and the bone fastener is permanently assembled.

In some embodiments, the spinal implant system comprises a modular system that includes a bone fastener including an array of members, such as, for example, bone screw shafts that can be selectively coupled to members, such as, for example, receivers. In some embodiments, the spinal implant system comprises a selectively coupled bone fastener that can be assembled on a surgical table or in-situ. In some embodiments, the selectively coupled bone fastener is assembled with a force of less than 50 Newtons (N). In some embodiments, the bone fastener is selectively coupled with a non-instrumented assembly. In some embodiments, the non-instrumented assembly comprises manually engaging a screw shaft with a head/receiver of the bone fastener. In some embodiments, the non-instrumented assembly comprises manually engaging the screw shaft in a pop-on engagement with the head/receiver of the bone fastener. In some embodiments, a force required to manually engage a screw shaft with a head/receiver of the bone fastener in a non-instrumented assembly is in a range of 2 to 50 N. In some embodiments, a force required to manually engage a screw shaft with a head/receiver of the bone fastener in a non-instrumented assembly is in a range of 5 to 10 N. In some embodiments, a screw shaft is manually engaged with a head/receiver of the bone fastener in a non-instrumented assembly, as described herein, such that removal of the head/receiver from the screw shaft requires a force and/or a pull-out strength of at least 5000 N. In some embodiments, this configuration provides manually engageable components of a bone fastener that are assembled without instrumentation, and subsequent to assembly, the assembled components have a selected pull-out strength and/or can be pulled apart, removed and/or separated with a minimum required force.

In some embodiments, the head assembly includes a ring disposed with an implant receiver connected with a screw shaft assembly. In some embodiments, the ring is configured to snap onto the screw shaft assembly. In some embodiments, the ring has a minimized thickness and/or height to facilitate snapping the ring onto the screw shaft assembly. In some embodiments, the force required to snap the ring onto the screw shaft assembly is in a range of 2 to 50 N. In some embodiments, the force required to snap the ring onto the screw shaft assembly is in a range of 5 to 10 N.

In some embodiments, the bone fastener is configured for assembly without the use of an instrument, such as, for example, a practitioner, surgeon and/or medical staff utilizes their hands for assembly. In some embodiments, the system requires minimal force to attach an implant receiver and a screw shaft assembly in-situ thereby reducing a pre-load on the vertebrae, such as, for, example, the pedicle. In some embodiments, the bone fastener includes an expandable ring.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed spinal implant system may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic, sacral and pelvic regions of a spinal column. The spinal implant system of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value; unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example; horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient; who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

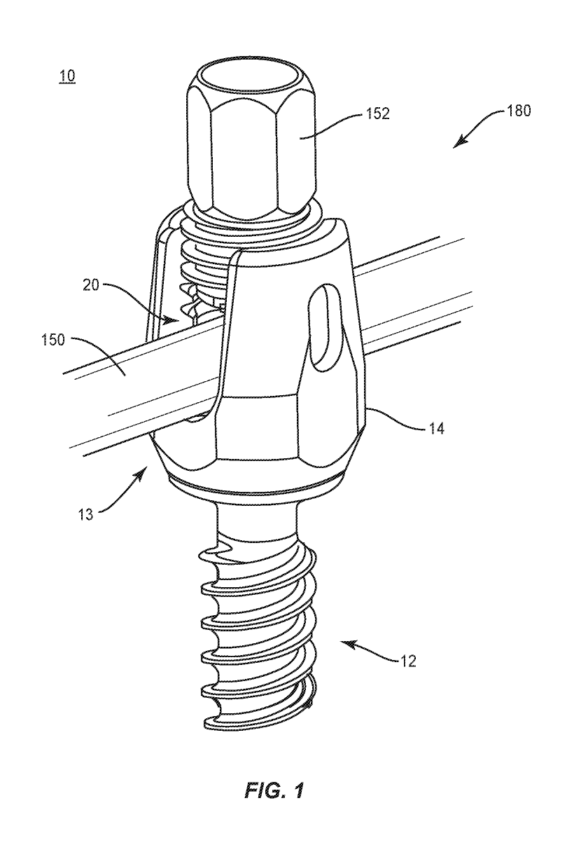

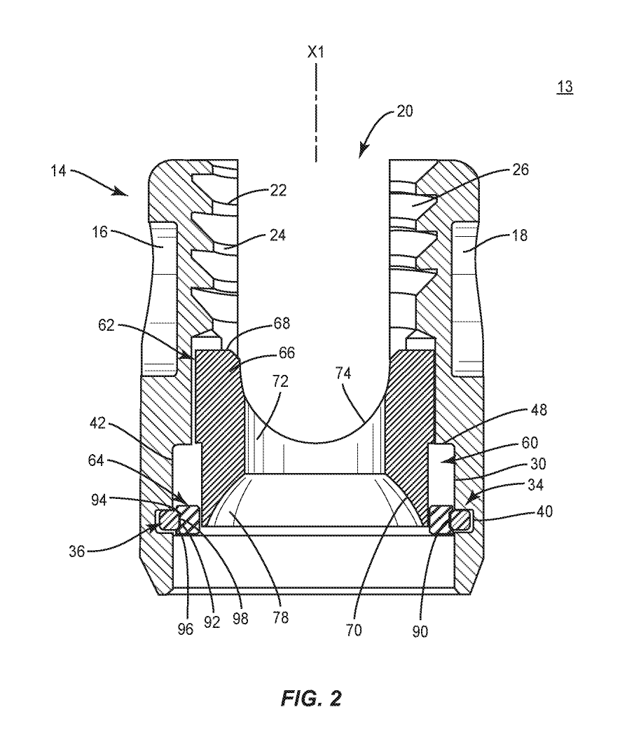

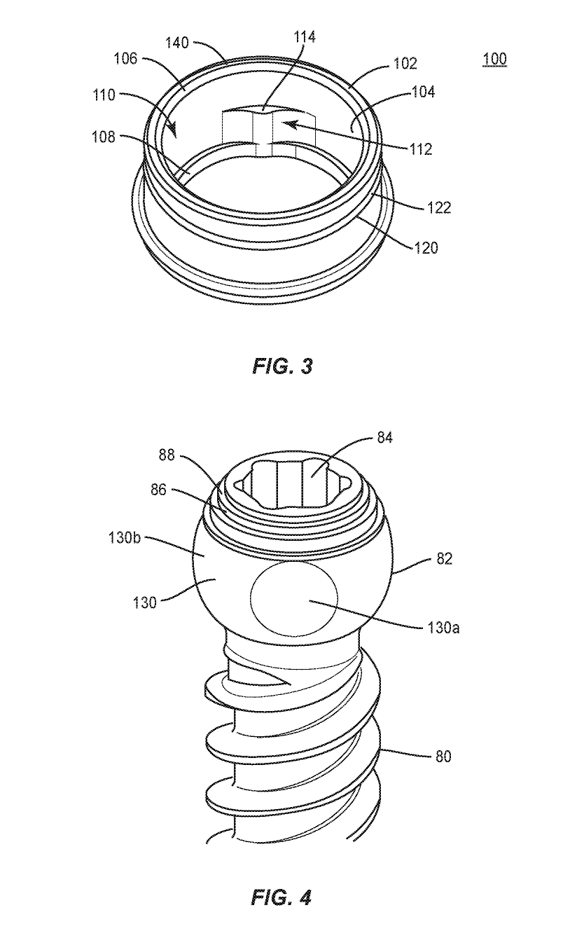

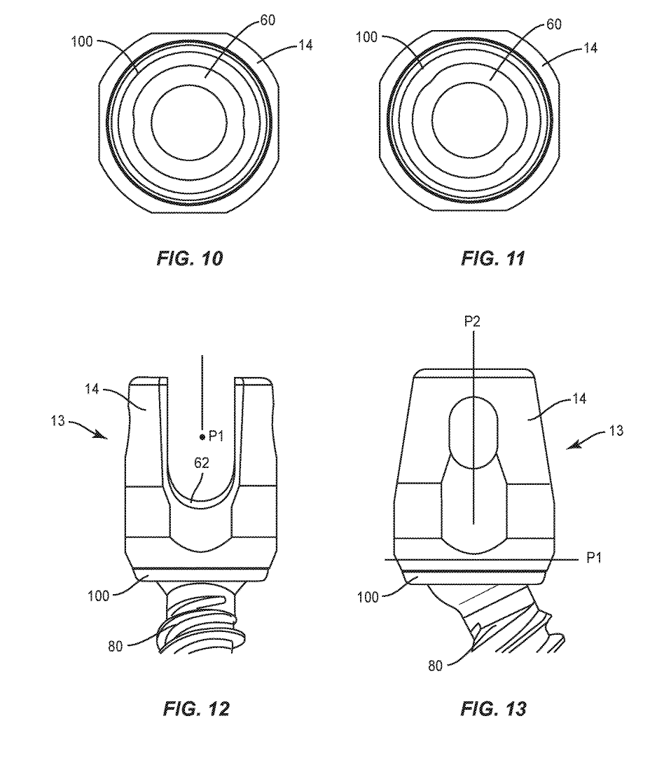

The following discussion includes a description of a surgical system including a bone fastener, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-11, there are illustrated components of a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys. Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyimide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

Spinal implant system 10 includes a spinal implant, such as, for example, a bone fastener 180. Bone fastener 180 comprises a member, such as, for example, a screw shaft assembly 12 and a member, such as, for example a head assembly 13. In some embodiments, screw shaft assembly 12 and head assembly 13 are assembled in situ or prior to implant to form bone fastener 180, as described herein.

Head assembly 13 includes a receiver 14. Receiver 14 extends along and defines an axis X1. Receiver 14 includes a pair of spaced apart arms 16, 18 that define an implant cavity 20 therebetween configured for disposal of a component of a spinal construct, such as, for example, a spinal rod 150.

Arms 16, 18 each extend parallel to axis X1. In some embodiments, arm 16 and/or arm 18 may be disposed at alternate orientations, relative to axis X1, such as, for example, transverse, perpendicular and/or other angular orientations such as acute or obtuse, coaxial and/or may be offset or staggered. Arms 16, 18 each include an arcuate outer surface extending between a pair of side surfaces. At least one of the outer surfaces and the side surfaces of arms 16, 18 have at least one recess or cavity therein configured to receive an insertion tool, compression instrument and/or instruments for inserting and tensioning bone fastener 180. In some embodiments, arms 16, 18 are connected at proximal and distal ends thereof such that receiver 14 defines a closed spinal rod slot.

Cavity 20 is substantially U-shaped. In some embodiments, all or only a portion of cavity 20 may have alternate cross section configurations, such as, for example, closed, V-shaped, W-shaped, oval, oblong triangular, square, polygonal, irregular, uniform, non-uniform, offset, staggered, and/or tapered. Receiver 14 includes an inner surface 22. A portion of surface 22 includes a thread form 24 located adjacent arm 16 and a thread form 26 located adjacent arm 18. Thread forms 24, 26 are each configured for engagement with a coupling member, such as, for example, a setscrew 152, to retain spinal rod 150 within cavity 20. In some embodiments, surface 22 may be disposed with the coupling member in alternate fixation configurations, such as, for example, friction fit, pressure fit, locking protrusion/recess, locking keyway and/or adhesive. In some embodiments, all or only a portion of surface 22 may have alternate surface configurations to enhance engagement with spinal rod 150 and/or setscrew 152, such as, for example, rough, arcuate, undulating, mesh, porous, semi-porous, dimpled and/or textured. In some embodiments, receiver 14 may include alternate configurations, such as, for example, closed, open and/or side access.

Receiver 14 includes a surface 30. Surface 30 defines a cavity, such as, for example, a groove 34 configured for disposal of a band, such as, for example, a circumferential ring 36. Groove 34 includes a circumferential channel 40 that accommodates expansion of ring 36. In some embodiments, ring 36 includes a selected height and a selected width. Ring 36 includes a circumference that extends between ends of ring 36. In some embodiments, the ends define an opening, such as, for example, a gap. In some embodiments, the gap is sized such that the gap has a thickness that is less than the height and the width. In some embodiments, upon disposal of ring 36 with groove 34, the surface of groove 34 resists and/or prevents axial translation of ring 36 relative to axis X1.

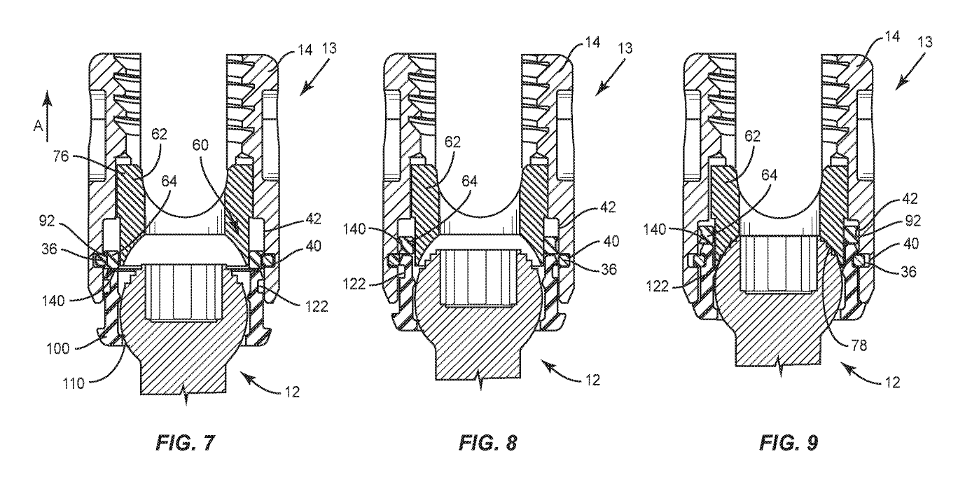

Ring 36 is expandable and resilient between a contracted and/or capture orientation, as shown in FIG. 9, and an expanded orientation, as shown in FIG. 8, as described herein. Ring 36 facilitates manual engagement of head assembly 13 and screw shaft assembly 12 such that a selected screw shaft assembly 12 is attached with head assembly 13 in a non-instrumented assembly, as described herein. In some embodiments, ring 36 is expandable and resilient between a contracted and/or capture orientation and an expanded orientation for assembly of screw shaft assembly 12 with head assembly 13, as shown and described for example with regard to FIGS. 7-9.

In some embodiments, surface 30 defines a slot 42. Slot 42 is configured for disposal of a part 60 and sleeve 64, as described herein. In some embodiments, slot 42 is separated from surface 22 by a protrusion, such as, for example, a lip 48. In some embodiments, lip 48 is configured as a stop surface to limit translation of sleeve 64. In some embodiments, all or only a portion of surface 22, 30 may have alternate surface configurations, such as, for example, rough, arcuate, undulating, mesh, porous, semi-porous, dimpled and/or textured.

Part 60 includes a crown 62 and a sleeve 64. Crown 62 is configured for disposal within cavity 20 and slot 42. Crown 62 includes a wall 66 having an end surface 68 and an end surface 70. Surface 68 is configured to define at least a portion 72 of cavity 20. Portion 72 is defined by an outer surface 74 that defines a curved portion of crown 62 configured for engagement with spinal rod 150. In some embodiments, all or only a portion of surface 74 may have alternate cross section configurations, such as, for example, oval, oblong triangular, square, polygonal, irregular, uniform, non-uniform, offset, staggered, and/or tapered. In some embodiments, a part, as described herein, may include a crown, a sleeve and/or other component of head assembly 13.

Surface 68 defines a receiver engagement portion, such as, for example, a flange 76 configured for mating engagement with a portion of surface 22. In some embodiments, flange 76 engages a portion of surface 22 in a keyed connection. In some embodiments, engagement of flange 76 and surface 22 prevents rotation and/or axial translation of crown 62 relative to surface 22 of receiver 14. Surface 74 is disposed in fixed alignment with surface 22 for disposal of spinal rod 150. Surface 70 defines an engagement portion 78 configured for engagement with a head 82 of a shaft 80 of head assembly 13, as described herein.

Sleeve 64 includes a surface 90 that defines a cavity, such as, for example, a groove 92. In some embodiments, groove 92 extends about all or a portion of surface 90. Groove 92 includes a surface 94 and a surface 96. Surface 94 is disposed at an angle relative to axis X1 to define a ramp. Surface 96 is disposed at an angle relative to axis X1 to define a ramp. The ramps of surface 94, 96 are oriented in spaced apart relation. An intermediate surface 98 is disposed between the ramps. Surface 98 is substantially even and circumferentially disposed about sleeve 64. In some embodiments, the ramps of surfaces 94, 96 are selectively inclined to resist and/or prevent displacement of ring 36 from channel 40 to provisionally fix sleeve 64 with receiver 14. In some embodiments, the inclination of the ramps of surfaces 94, 96 facilitate disengagement of ring 36 from groove 92 upon axial translation of sleeve 64, as described herein. In some embodiments, surfaces 94, 96 are oriented substantially perpendicular to axis X1. In some embodiments, groove 92 does not include inclined surfaces, as described above, and alternatively includes a protrusion or a lip configured to engage ring 36.

Sleeve 64 is configured for translation within slot 42 along surface 30. Sleeve 64 is configured for translation relative to crown 62 and receiver 14 within slot 42. Translation of sleeve 64 within slot 42 moves sleeve 64 between a first configuration, as shown in FIG. 7, such that ring 36 is disposed within channel 40 and groove 92 to provisionally fix sleeve 64 relative to receiver 14 and a second configuration, as shown in FIG. 9, such that ring 36 remains disposed within channel 40 and a base 100 to fix head assembly 13 with screw shaft assembly 12, as described herein.

For example, as sleeve 64 is engaged with base 100 and translates, in a direction shown by arrow A in FIG. 7, the ramp of surface 96 engages ring 36 such that surface 96 slides over ring 36 to release sleeve 64 from ring 36. Surface 96 expands ring 36 into channel 40, as shown in FIG. 8. Expansion of ring 36 facilitates axial translation of screw shaft assembly 12 into slot 42, as described herein.

Screw shaft assembly 12 includes shaft 80 and head 82. Shaft 80 is configured to penetrate tissue, such as, for example, bone. In some embodiments, shaft 80 includes an outer surface having an external thread form. In some embodiments, the external thread form may include a single thread turn or a plurality of discrete threads. Head 82 includes a tool engaging portion 84 configured to engage a surgical tool or instrument, as described herein. In some embodiments, portion 84 includes a hexagonal cross-section to facilitate engagement with a surgical tool or instrument, as described herein. In some embodiments, portion 84 may have alternative cross-sections, such as, for example, rectangular, polygonal, hexalobe, oval, or irregular. In some embodiments, head 82 includes a surface 86 that defines a plurality of ridges 88 to improve purchase of head 82 with crown 62. In some embodiments, head 82 includes an outer surface 130. In some embodiments, surface 130 includes planar surfaces, such as, for example, flats 130a and arcuate surfaces 130b. Head 82 is configured for attachment with base 100, as described herein.

Screw shaft assembly 12 includes base 100, which has a wall 102. Wall 102 includes a surface 104. Surface 104 includes circumferential inner flanges 108. In some embodiments, flange 106 is spaced apart from flange 108. Flanges 106, 108 and surface 104 define a cavity 110 configured for disposal of head 82. Surface 104 facilitates engagement of head 82 with base 100 via a pressure and/or force fit connection.

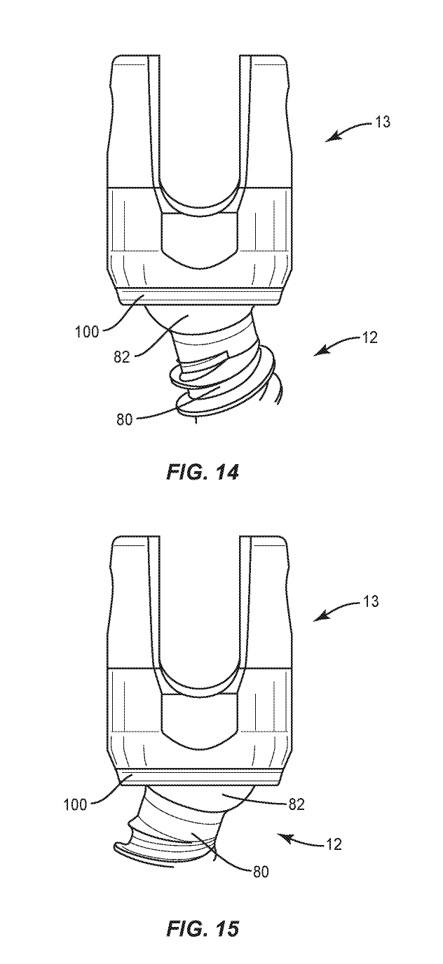

In some embodiments, surface 104 includes mating elements, such as, for example, an index elements 112. In some embodiments, index elements 112 include a protrusion 114. Protrusion 114 is configured to interface flats 130a such that shaft 80 is limited to rotation about a single axis disposed in a plane P1 relative to receiver 14, as shown FIGS. 12-19. In some embodiments, shaft 80 is rotatable relative to receiver 14 in plane P1. Rotation of shaft 80 in plane P2 is resisted and/or prevented due to the interface of flats 130a with index elements 112 such that assembly of screw shaft assembly 12 and head assembly 13 forms a uni-planar bone fastener 180. In some embodiments, plane P1 is disposed along a rod slot plane and plane P2 is oriented substantially orthogonal, as shown in FIGS. 12 and 15.

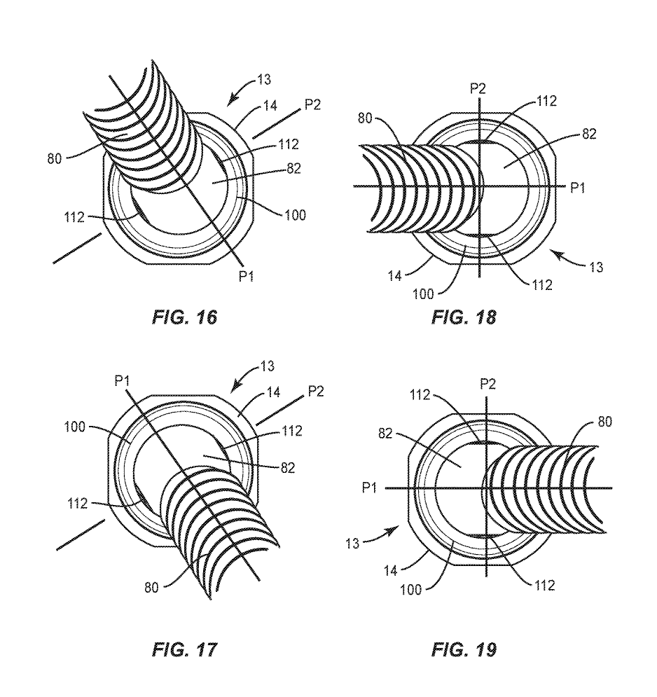

In some embodiments, base 100 may be disposed with head 82 in alternate fixation configurations, such as, for example, friction fit, pressure fit, locking protrusion/recess, locking keyway and/or adhesive. Base 100 is configured for rotation relative to head 82. In some embodiments, base 100 is configured for rotation in range of 360 degrees relative to head 82 to facilitate positioning of shaft 80 with tissue. In some embodiments, base 100 is configured for selective rotation in range of 360 degrees relative to and about head 82 such that shaft 80 is selectively aligned for rotation in a plane P1 relative to receiver 14, and flats 130a engage index elements 112 to resist and/or prevent rotation of shaft 80 in plane P2, as shown in FIGS. 16-19.

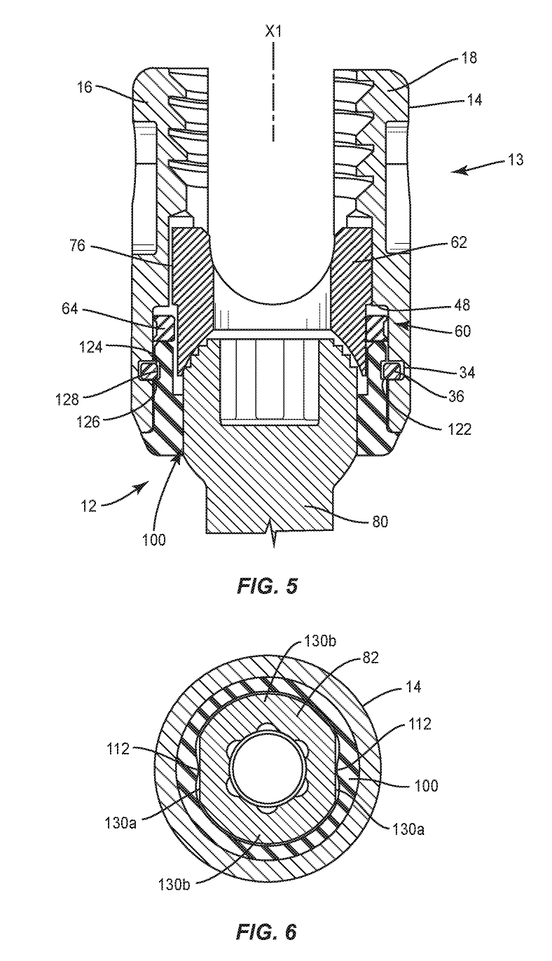

Wall 102 includes a surface 120. Surface 120 defines a cavity, such as, for example, a groove 122. Groove 122 includes a surface 124 and a surface 126. Surfaces 124, 126 are oriented substantially perpendicular to axis X1. In some embodiments, surfaces 124, 126 may be disposed in various orientations and/or inclinations, such as, for example, transverse and/or at angular orientations, such as acute or obtuse relative to axis X1. Surface 124 is oriented in spaced apart relation relative to surface 126. An intermediate surface 128 is disposed between surfaces 124, 126. Surface 128 is substantially even and circumferentially disposed about groove 122. In some embodiments, surfaces 124, 126, 128 may be disposed about all or only a portion of groove 122. Groove 122 is configured for disposal of ring 36 to prevent displacement of ring 36 from channel 40 and to permanently fix head assembly 13 with screw shaft assembly 12, as shown in FIG. 5. Base 100 is configured for axial translation relative to head assembly 13 within slot 42, as described herein.

For example, base 100 is assembled with head 82 and a surface 140 engages sleeve 64 to release sleeve 64 from ring 36, as described herein with regard to FIG. 7. With ring 36 expanded into channel 40, as shown in FIG. 8, base 100 and head 82 axially translate relative to head assembly 13 within slot 42 to align groove 122 with channel 40. Alignment of groove 122 with channel 40 allows ring 36 to resiliently contract to the capture orientation, as shown in FIG. 9, for disposal of ring 36 within groove 122 and channel 40. Ring 36 is fixed within channel 40 and groove 122. Surfaces 124, 126 resist and/or prevent disengagement of ring 36 from channel 40 and groove 122 to permanently assemble screw shaft assembly 12 with head assembly 13.

In some embodiments, screw shaft assembly 12 is manually engageable with head assembly 13 and/or base 100 is coupled with receiver 14 in a non-instrumented assembly such that ring 36 translates from disposal with channels 40, 94 and into channel 40 and groove 122, as described herein. In some embodiments, manual engagement and/or non-instrumented assembly of head assembly 13 and screw shaft assembly 12 includes coupling without use of separate and/or independent instrumentation engaged with screw shaft assembly 12 components to effect assembly. In some embodiments, manual engagement and/or non-instrumented assembly includes a practitioner; surgeon and/or medical staff grasping head assembly 13 and screw shaft assembly 12 and forcibly assembling the components. In some embodiments, manual engagement and/or non-instrumented assembly includes a practitioner, surgeon and/or medical staff grasping head assembly 13 and screw shaft assembly 12 and forcibly snap fitting the components together, as described herein. In some embodiments, manual engagement and/or non-instrumented assembly includes a practitioner, surgeon and/or medical staff grasping head assembly 13 and screw shaft assembly 12 and forcibly pop fitting the components together and/or pop fitting head assembly 13 onto screw shaft assembly 12, as described herein. In some embodiments; a force in a range of 2-50 N is required to manually engage head assembly 13 and screw shaft assembly 12 and forcibly assemble the components. For example, a force in a range of 2-50 N is required to snap fit and/or pop fit assemble head assembly 13 and screw shaft assembly 12. In some embodiments, a force in a range of 5-10 N is required to manually engage head assembly 13 and screw shaft assembly 12 and forcibly assemble the components. For example, a force in a range of 5-10 N is required to snap fit and/or pop fit assemble head assembly 13 and screw shaft assembly 12. In some embodiments, screw shaft assembly 12 is manually engaged with head assembly 13 in a non-instrumented assembly, as described herein, such that removal of head assembly 13 and screw shaft assembly 12 requires a force and/or a pull-out strength of at least 5000 N. In some embodiments, this configuration provides manually engageable components that are assembled without instrumentation, and subsequent to assembly, the assembled components have a selected pull-out strength and/or can be pulled apart, removed and/or separated with a minimum required force.

In some embodiments, spinal implant system 10 comprises a spinal implant kit, as described herein, which includes a plurality of screw shaft assemblies 12. Screw shaft assembly 12 is configured for selection from the plurality of screw shaft assemblies such that screw shaft assembly 12 is connectable with an interchangeable member, such as, for example, a head assembly 13.

In assembly, operation and use, spinal implant system 10, similar to the systems and methods described herein, includes a screw shaft assembly 12 for connection with a head assembly 13, and is employed with a surgical procedure for treatment of a spinal disorder affecting a section of a spine of a patient, as discussed herein. Spinal implant system 10 is employed with a surgical procedure for treatment of a condition or injury of an affected section of the spine.

In some embodiments, spinal implant system 10 includes a selected screw shaft assembly 12 for connection with an interchangeable head assembly 13 having a selected movement, as described herein. As such, the components of spinal implant system 10 include a spinal implant kit, which comprises a plurality of screw shaft assemblies 12 and interchangeable head assembly 13. In some embodiments, spinal implant system 10 includes a spinal implant kit, which comprises a plurality of screw shaft assemblies 12 and a compatible head assembly 13. The plurality of screw shaft assemblies include screw shaft assembly 12 and alternate screw shaft assemblies, such as those described herein, that interface with interchangeable head assembly 13 to comprise one or more bone fastener configurations. Selected screw shaft assembly 12 and one or a plurality of spinal implants, such as, for example, spinal rods 150 can be delivered or implanted as a pre-assembled device or can be assembled in situ. The components of spinal implant system 10 may be may be completely or partially revised, removed or replaced.

In some embodiments, a screw shaft assembly 12 is selected from the kit of the plurality of screw shaft assemblies 12 for interchangeable connection with head assembly 13 to comprise a bone fastener 180 having a selected movement. In some embodiments, the kit of screw shaft assemblies 12 includes a variety of screw shaft assemblies having different movement configurations when assembled with an interchangeable head assembly 13, such as, for example, multi-axial movement, sagittal angulation movement, fixed axis movement, mono-axial movement and/or uni-planar movement.

In some embodiments, head assembly 13 includes receiver 14 assembled with crown 60, sleeve 64 and ring 36, as described herein. In some embodiments, ring 36 is disposed with channel 40 and groove 92 in a contracted and/or capture orientation to provisionally resist and/or prevent displacement of ring 36 from channel 40, as shown in FIG. 7. Ring 36 is configured to provisionally fix sleeve 64 relative to receiver 14 forming head assembly 13.

The selected screw shaft assembly 12 is manually engageable, as described herein, with head assembly 13, as shown in FIGS. 7-9. Base 100 is assembled with head 82. Screw shaft assembly 12 is engaged with head assembly 13 such that surface 140 engages sleeve 64. Base 100 engages sleeve 64 such that sleeve 64 translates, in a direction shown by arrow A in FIG. 7. The ramp of surface 96 engages ring 36 such that surface 96 slides over ring 36 to release sleeve 64 from ring 36. Surface 96 expands ring 36 into channel 40, as shown in FIG. 8.

With ring 36 expanded into channel 40, base 100 and head 82 axially translate relative to head assembly 13 within slot 42 to align groove 122 with channel 40. Alignment of groove 122 with channel 40 allows ring 36 to resiliently contract to the capture orientation, as shown in FIG. 9, for disposal of ring 36 within groove 122 and channel 40. Ring 36 is fixed within channel 40 and groove 122. Surfaces 124, 126 resist and/or prevent disengagement of ring 36 from channel 40 and groove 122 to permanently assemble screw shaft assembly 12 with head assembly 13 in a configuration to form bone fastener 180 for use with a surgical procedure, as described herein.

In use, for treatment of a spinal disorder, bone fastener 180 including assembled screw shaft assembly 12 and head assembly 13 can be threaded and engaged with tissue. In some embodiments, the selected bone fastener 180 is disposed adjacent vertebrae at a surgical site and is manipulated to drive, torque, insert or otherwise connect shaft 80 with vertebrae in connection with a surgical procedure, as described herein.

In some embodiments, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the fixation elements with vertebrae. In some embodiments, the agent may be HA coating. In some embodiments, the agent may include one or a plurality of therapeutic agents and/or pharmacological agents for release, including sustained release, to treat, for example, pain, inflammation and degeneration.

In some embodiments, the use of microsurgical and image guided technologies may be employed to access, view and repair spinal deterioration or damage, with the aid of spinal implant system 10. The components of spinal implant system 10 can be made of radiolucent materials such as polymers. Radiomarkers may be included for identification under x-ray, fluoroscopy, CT or other imaging techniques.

In some embodiments, spinal implant system 10 can include one or a plurality of bone fasteners 180 such as those described herein and/or fixation elements, which may be employed with a single vertebral level or a plurality of vertebral levels. In some embodiments, bone fasteners 180 may be engaged with vertebrae in various orientations, such as, for example, series, parallel, offset, staggered and/or alternate vertebral levels. In some embodiments, bone fasteners 180 may be configured as multi-axial screws, sagittal angulation screws, pedicle screws, mono-axial screws, uni-planar screws, fixed screws, anchors, tissue penetrating screws, conventional screws, expanding screws. In some embodiments, bone fasteners 180 may be employed with wedges, anchors, buttons, clips, snaps, friction fittings, compressive fittings, expanding rivets, staples, nails, adhesives, posts, connectors, fixation plates and/or post.

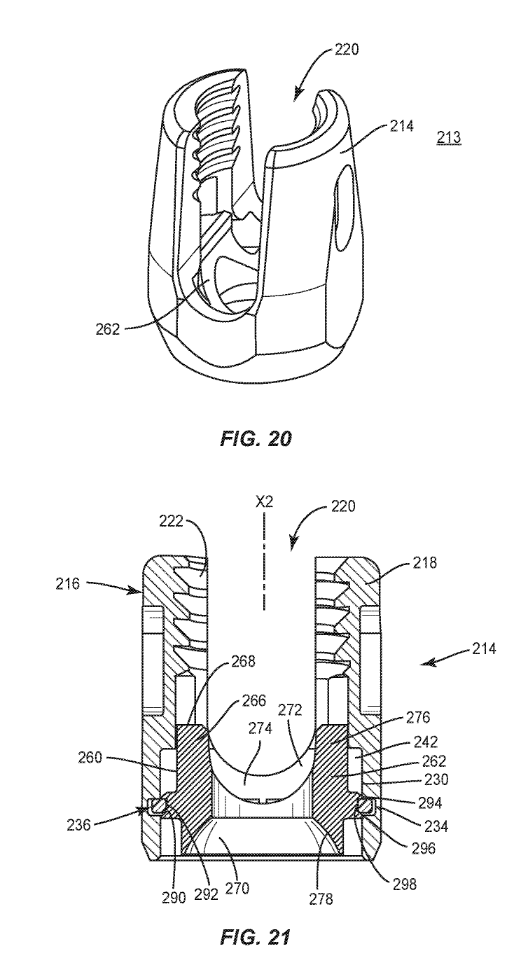

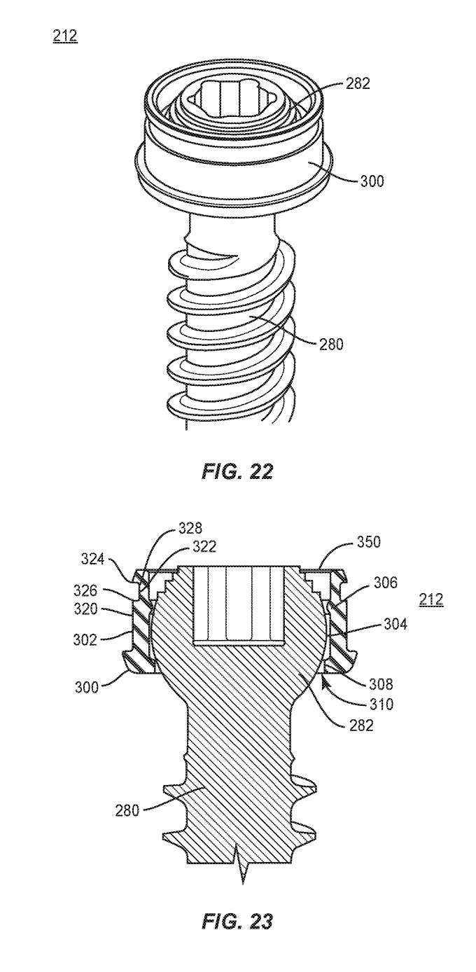

In one embodiment, as shown in FIGS. 20-34, spinal correction system 10, similar to the systems and methods described above with regard to FIGS. 1-19, includes a bone fastener 380, similar to bone fastener 180. Bone fastener 380 comprises a member, such as, for example, a screw shaft assembly 212, similar to screw shaft assembly 12 described herein, and a member, such as, for example a head assembly 213, similar to head assembly 13 described herein.

Head assembly 213 includes a receiver 214, similar to receiver 14 described herein. Receiver 214 extends along and defines an axis X2. Receiver 214 includes a pair of spaced apart arms 216, 218 that define an implant cavity 220 therebetween configured for disposal of a component of a spinal construct, such as, for example, a spinal rod (not shown) and similar to that described herein.

Receiver 214 includes a surface 230. Surface 230 defines a cavity, such as, for example, a groove 234 configured for disposal of a band, such as, for example, a circumferential ring 236, similar to ring 36 described herein. Groove 234 includes a circumferential channel 240 that accommodates expansion of ring 236. Ring 236 is expandable and resilient between a contracted and/or capture orientation, as shown in FIG. 26, and an expanded orientation, as shown in FIG. 25. Ring 236 facilitates manual engagement of head assembly 213 and screw shaft assembly 212 such that a selected screw shaft assembly 212 is attached with head assembly 213 in a non-instrumented assembly, as described herein.

Surface 230 defines a slot 242, which is configured for disposal of a part 260, similar to part 60 described herein. Slot 242 is separated from surface 222 by a protrusion, such as, for example, a lip 248. In some embodiments, lip 248 is configured as a stop surface to limit translation of part 260. Part 260 includes a crown 262, which is configured for disposal within cavity 220 and slot 242.

Crown 262 includes a wall 266 having an end surface 268 and an end surface 270. Surface 268 is configured to define at least a portion 272 of cavity 220. Portion 272 is defined by an outer surface 274 that defines a curved portion of crown 262 configured for engagement with the spinal rod. Surface 268 defines a receiver engagement portion 276 configured for mating engagement with a portion of surface 222. Surface 274 is disposed in fixed alignment with surface 222 for disposal of a spinal rod. Surface 270 defines an engagement portion 278 configured for engagement with a head 282 of a shaft 280 of head assembly 213, as described herein.

Crown 262 includes a surface 290 that defines a cavity, such as, for example, a groove 292. In some embodiments, groove 292 extends about all or a portion of surface 290. Groove 292 includes a surface 294 and a surface 296. Surface 294 is disposed at an angle relative to axis X2 to define a ramp. Surface 296 is disposed at an angle relative to axis X2 to define a ramp. The ramps of surfaces 294, 296 are oriented in spaced apart relation. An intermediate surface 298 is disposed between the ramps. Surface 298 is substantially even and circumferentially disposed about crown 262. In some embodiments, the ramps of surfaces 294, 296 are selectively inclined to resist and/or prevent displacement of ring 236 from channel 240 to provisionally fix crown 262 with receiver 214. In some embodiments, the inclination of the ramps of surfaces 294, 296 facilitate disengagement of ring 236 from groove 292 upon axial translation of crown 262, as described herein.

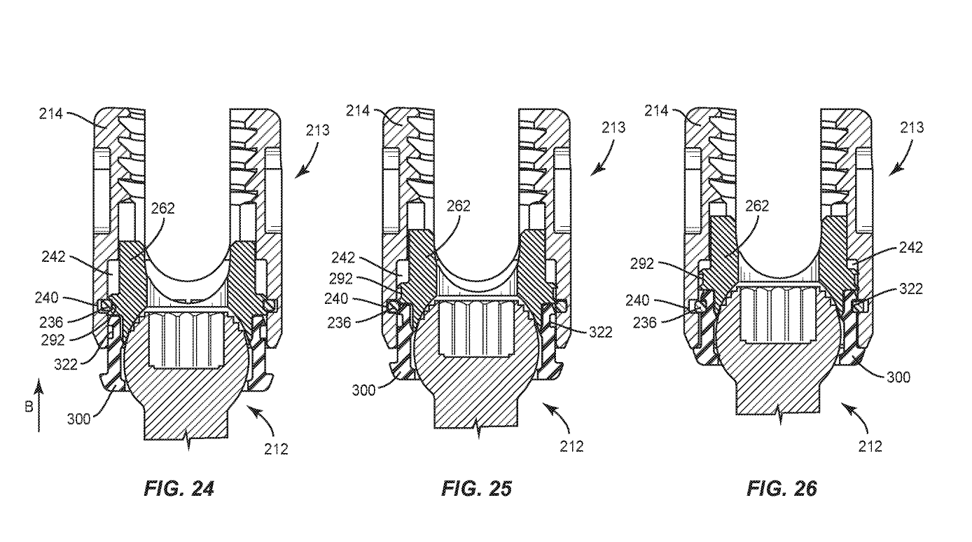

Crown 262 is configured for translation within slot 242 along surface 230. Translation of crown 262 within slot 242 moves crown 262 between a first configuration, as shown in FIG. 24, such that ring 236 is disposed within channel 240 and groove 292 to provisionally fix crown 262 relative to receiver 214 and a second configuration, as shown in FIG. 26, such that ring 236 is disposed within channel 240 and a base 300 to fix head assembly 213 with screw shaft assembly 212, as described herein.

For example, as crown 262 is engaged with base 300 and translates, in a direction shown by arrow B in FIG. 24, the ramp of surface 296 engages ring 236 such that surface 296 slides over ring 236 to release crown 262 from ring 236. Surface 296 expands ring 236 into channel 240, as shown in FIG. 25. Expansion of ring 236 facilitates axial translation of screw shaft assembly 212 into slot 242, as described herein.

Screw shaft assembly 212 includes shaft 280 and head 282, similar to shaft 80, head 82, described herein. Head 282 is slidably engageable with base 300, as described herein, and movable relative to head assembly 213 such that shaft 280 is rotatable along a plurality of axes and/or multi-axial relative to head assembly 213 including rotation about axis X2. Head 282 is configured for attachment with base 300, as described herein.

Screw shaft assembly 212 includes base 300, which has a wall 302. Wall 302 includes a surface 304. Surface 304 includes circumferential inner flanges 306, 308 that are spaced apart. Flanges 306, 308 and surface 304 define a cavity 310 configured for disposal of head 282. Surface 304 facilitates engagement of head 282 with base 300 via a pressure and/or force fit connection.

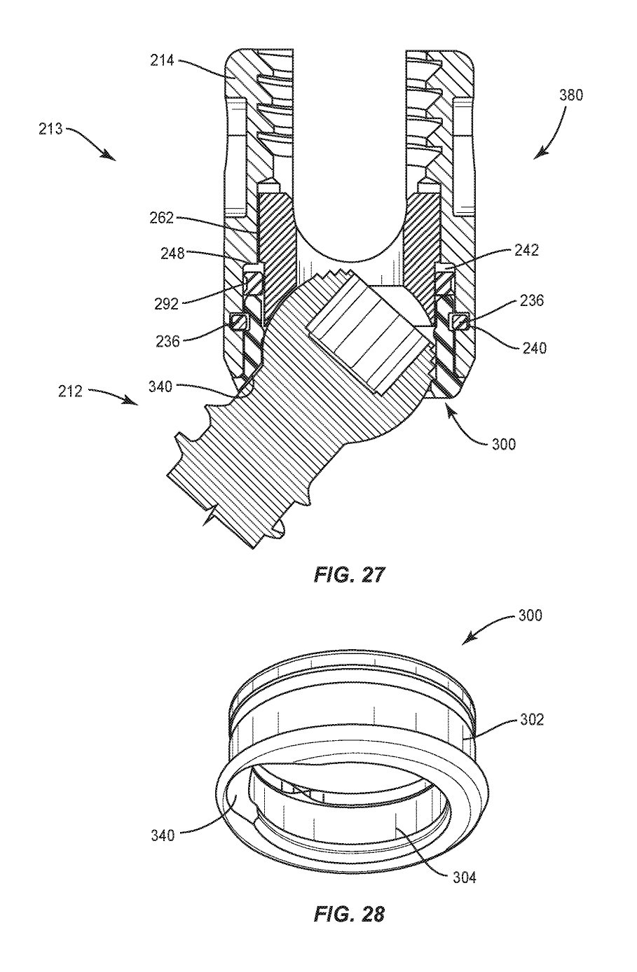

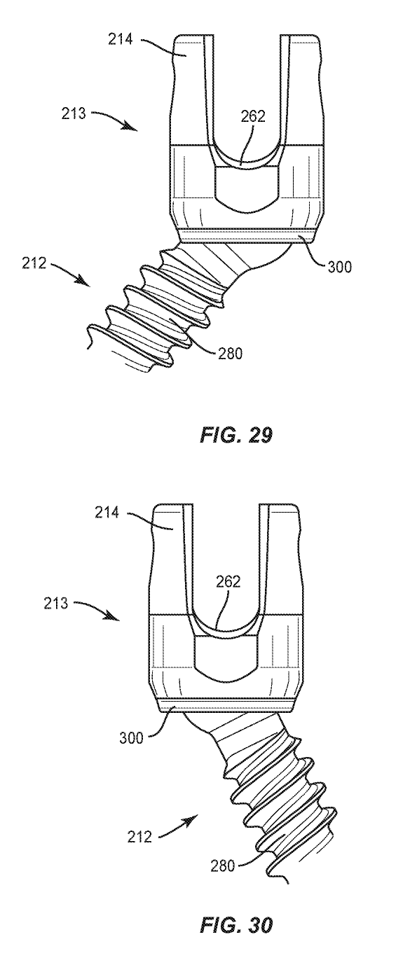

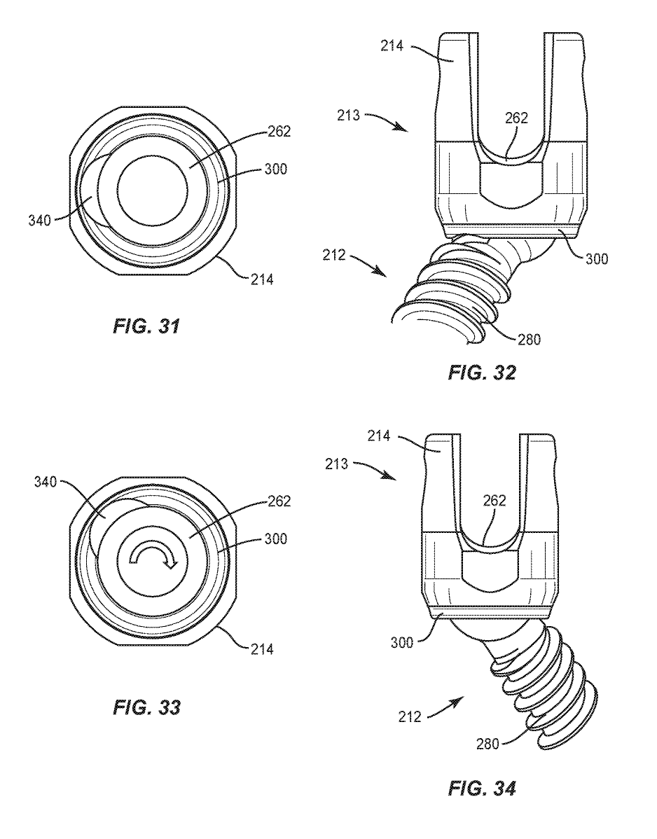

Surface 304 is configured to interface head 282 such that shaft 280 is rotatable about multiple axes relative to receiver 214, as shown FIGS. 29-34. In some embodiments, shaft 280 is rotatable in one or a plurality of planes relative to receiver 214, similar to that described herein, and/or rotatable about axis X2 in an angular range of 360 degrees, for example, as shown in FIGS. 31 and 33. In some embodiments, assembly of screw shaft assembly 212 and head assembly 213 forms a multi-axial bone fastener 380. In some embodiments, as shown in FIGS. 27-34, wall 302 defines a lateral recess 340 configured for disposal of shaft 280. Recess 340 is concavely curved such that shaft 280 is disposable therein for movement of shaft 280 in a greater angular range of motion relative to axis X2 and facilitates a biased angle configuration.

In some embodiments, base 300 may be disposed with head 282 in alternate fixation configurations, such as, for example, friction fit, pressure fit, locking protrusion/recess, locking keyway and/or adhesive. Base 300 is configured for rotation relative to head 282. In some embodiments, base 300 is configured for rotation in range of 360 degrees relative to head 282 to facilitate positioning of shaft 280 with tissue. In some embodiments, base 300 is configured for selective rotation in range of 360 degrees relative to and about head 282 such that shaft 280 is selectively aligned for rotation relative to receiver 214, as shown in FIGS. 31-34.

Wall 302 includes a surface 320. Surface 320 defines a cavity, such as, for example, a groove 322. Groove 322 includes a surface 324 and a surface 326. Surfaces 324, 326 are oriented substantially perpendicular to axis X2. In some embodiments, surfaces 324, 326 may be disposed in various orientations and/or inclinations, such as, for example, transverse and/or at angular orientations, such as acute or obtuse relative to axis X2. Surface 324 is oriented in spaced apart relation relative to surface 326. An intermediate surface 328 is disposed between surfaces 324, 326. Surface 328 is substantially even and circumferentially disposed about groove 322. In some embodiments, surfaces 324, 326, 328 may be disposed about all or only a portion of groove 322. Groove 322 is configured for disposal of ring 236 to resist and/or prevent displacement of ring 236 from channel 240 and to permanently fix head assembly 213 with screw shaft assembly 212, as shown in FIG. 27. Base 300 is configured for axial translation relative to head assembly 213 within slot 242, as described herein.

For example, base 300 is assembled with head 282 and a surface 350 engages crown 262 to release crown 262 from ring 236, as described herein with regard to FIG. 24. With ring 236 expanded into channel 240, as shown in FIG. 25, base 300 and head 282 axially translate relative to head assembly 213 within slot 242 to align groove 322 with channel 240. Alignment of groove 322 with channel 240 allows ring 236 to resiliently contract to the capture orientation, as shown in FIG. 26, for disposal of ring 236 within groove 322 and channel 240. Ring 236 is fixed within channel 240 and groove 322. Surfaces 324, 326 resist and/or prevent disengagement of ring 236 from channel 240 and groove 322 to permanently assemble screw shaft assembly 212 with head assembly 213. In some embodiments, screw shaft assembly 212 is manually engageable with head assembly 213 and/or base 300 is coupled with receiver 214 in a non-instrumented assembly such that ring 236 translates from disposal with channels 240, 294 and into channel 240 and groove 322, similar to that described herein.

In some embodiments, spinal implant system 10 comprises a spinal implant kit, similar to that described herein, which includes a plurality of screw shaft assemblies 212. Screw shaft assembly 212 is configured for selection from the plurality of screw shaft assemblies such that screw shaft assembly 212 is connectable with an interchangeable member, such as, for example, a head assembly 213.

In assembly, operation and use, spinal implant system 10, similar to the systems and methods described herein, includes a screw shaft assembly 212 for connection with a head assembly 213, and is employed with a surgical procedure for treatment of a spinal disorder affecting a section of a spine of a patient, as discussed herein. Head assembly 213 includes receiver 214 assembled with crown 260 and ring 236, as described herein. Ring 236 is disposed with channel 240 and groove 292 in a contracted and/or capture orientation to provisionally resist and/or prevent displacement of ring 236 from channel 240, as shown in FIG. 24. Ring 236 is configured to provisionally fix crown 262 relative to receiver 214 forming head assembly 213.

Screw shaft assembly 212 is manually engageable, as described herein, with head assembly 213, as shown in FIGS. 24-26. Base 300 is assembled with head 282. Screw shaft assembly 212 is engaged with head assembly 213 such that surface 230 engages crown 262. Base 300 engages crown 262 such that crown 262 translates, in a direction shown by arrow B in FIG. 24. The ramp of surface 296 engages ring 236 such that surface 296 slides over ring 236 to release crown 262 from ring 236. Surface 296 expands ring 236 into channel 40, as shown in FIG. 25.

With ring 236 expanded into channel 240, base 300 and head 282 axially translate relative to head assembly 213 within slot 242 to align groove 322 with channel 240. Alignment of groove 322 with channel 240 allows ring 236 to resiliently contract to the capture orientation, as shown in FIG. 26, for disposal of ring 236 within groove 322 and channel 240. Ring 236 is fixed within channel 240 and groove 322. Surfaces 324, 326 resist and/or prevent disengagement of ring 236 from channel 240 and groove 322 to permanently assemble screw shaft assembly 212 with head assembly 213 in a configuration to form bone fastener 380 for use with a surgical procedure, similar to that described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. A bone fastener comprising:
a first member defining an implant cavity and a groove configured for disposal of a band,
the first member including a part disposed with the implant cavity and engageable with the band to provisionally fix the part relative to the first member; and
a second member configured to penetrate tissue and including a base that moves the part and engages the band to connect the members,
wherein the base includes at least one mating element that interfaces with a head of the second member to resist and/or prevent rotation of the second member relative to the first member.

2. A bone fastener as recited in claim 1, wherein the base is manually engageable with the part to connect the members in a non-instrumented assembly.

3. A bone fastener as recited in claim 1, wherein the members are engageable in a snap-fit assembly.

4. A bone fastener as recited in claim 1, wherein the members are engageable in a pop-on assembly.

5. A bone fastener as recited in claim 1, wherein the base is attached with the head of the second member via a pressure fit.

6-8. (canceled)

9. A bone fastener as recited in claim 1, wherein the base defines an outer groove for disposal of the band to connect the members.

10. A bone fastener as recited in claim 1, wherein the part includes a crown that defines an outer groove for disposal of the band to provisionally fix the crown relative to the first member.

11. A bone fastener as recited in claim 1, wherein the part includes a sleeve that defines an outer groove for disposal of the band to provisionally fix the sleeve relative to the first member.

12. (canceled)

13. A bone fastener as recited in claim 1, wherein the band is expandable within the groove and the base is manually engageable with the band to connect the members.

14. A bone fastener as recited in claim 13, wherein the band is expandable between a capture orientation and an expanded orientation.

15. A bone fastener as recited in claim 1, wherein the band is a circumferential ring that defines a gap.

16. A bone fastener as recited in claim 1, wherein the base is axially translatable relative to the first member to displace the part from engagement with the band.

17. A method of assembly for a bone fastener, the method comprising the steps of:
providing the bone fastener recited in claim 1;
engaging the part with the band to provisionally fix the part relative to the first member; and
manually engaging the base with the part to move the part and engage the band to connect the first member with the second member to comprise a bone fastener in a non-instrumented assembly.

18. (canceled)

19. A method as recited in claim 17, wherein the step of manually engaging includes snap fitting the second member with the first member.

20. (canceled)

21. A bone fastener comprising:
a first member defining an implant cavity and a groove configured for disposal of a band,
the first member including a part disposed with the implant cavity and engageable with the band to provisionally fix the part relative to the first member; and
a second member configured to penetrate tissue and including a base that moves the part and engages the band to connect the members,
the base includes at least one mating element that interfaces with a head of the second member such that the second member is rotatable about a single axis relative to the first member.

22. A bone fastener as recited in claim 21, wherein the base is manually engageable with the part to connect the members in a non-instrumented assembly.

23. A bone fastener as recited in claim 21, wherein the members are engageable in a snap-fit assembly.

24. A bone fastener as recited in claim 21, wherein the members are engageable in a pop-on assembly.

25. A bone fastener as recited in claim 21, wherein the base is attached with the head of the second member via a pressure fit.

26. A bone fastener as recited in claim 21, wherein the part includes a crown that defines an outer groove for disposal of the band to provisionally fix the crown relative to the first member.
